(19) United States
(12) Patent Application Publication
(10) Pub. No.: US 2013/0294613 A1
(43) Pub. Date: Nov. 7, 2013

(54) ELECTROLARYNX DEVICES AND USES THEREOF

(71) Applicants: George Nagel, Medford, MA (US); Jael Jose, Somerville, MA (US); Nicholas Matiasz, Somerville, MA (US); Karen Panetta, Rockport, MA (US)

(72) Inventors: George Nagel, Medford, MA (US); Jael Jose, Somerville, MA (US); Nicholas Matiasz, Somerville, MA (US); Karen Panetta, Rockport, MA (US)

(21) Appl. No.: 13/812,568

(22) PCT Filed: Jul. 28, 2011
(86) PCT No.: PCT/US11/45700

§ 371 Date: Jul. 26, 2013

Related U.S. Application Data
(60) Provisional application No. 61/368,472, filed on Jul. 28, 2010.

(51) Int. Cl. A61F 2/20

(52) U.S. Cl. CPC A61F 2/20
USPC 381/70

(57) ABSTRACT

The present invention relates to electrolarynx devices and their use. In particular, the present invention relates to methods and compositions (e.g., devices) to provide electrolarynx (EL) users with greater intonation in their speech.

This application claims priority to provisional application 61/368,472, filed Jul. 28, 2010, which is herein incorporated by reference in its entirety.

### FIELD OF THE INVENTION

The present invention relates to electrolarynx devices and their use. In particular, the present invention relates to methods and compositions (e.g., devices) to provide electrolarynx (EL) users with greater intonation in their speech.

### BACKGROUND

Normal human speech is in part facilitated by the larynx, an organ of the vocal tract that helps to control the pitch and volume of the voice. When a patient's larynx must be surgically removed—often due to laryngeal cancer—the laryngectomee loses the ability to speak in the usual manner. Electrolarynx (EL) devices are often used by such patients to communicate; these medical instruments act as artificial larynxes by producing the mechanical vibration necessary to excite the remaining vocal tract. The sound waves that are produced by this vibration are then articulated by the teeth, tongue, and lips.

Audible speech is produced by this method, but EL speech is far less intelligible than normal human speech. Rather than using the larynx as the sound source, EL speech uses a crude, buzzing diaphragm, which does not produce a waveform with the same acoustic characteristics that are present in a human voice. This diaphragm, which is held against the neck so that the mechanical vibration is transmitted to the vocal tract, produces a sound that is neither pleasant nor particularly clear.

There is a great need to improve current EL designs so that laryngectomees can communicate with a level of expression and intelligibility that is enjoyed by the normal population.

### SUMMARY

The present invention relates to electrolarynx devices and their use. In particular, the present invention relates to methods and compositions (e.g., devices) to provide electrolarynx (EL) users with greater intonation in their speech. For example, embodiments of the present invention provide an electric artificial larynx device and methods of using said device to generate speech (e.g., in a subject lacking a functional larynx), comprising: a) a user interface for selecting a volume and a frequency, wherein the frequency is selected across a frequency range; b) a pulse generator circuit that translates the volume and frequency into a voltage signal; and c) a sound source unit comprising a diaphragm that translates the voltage signal into sound (e.g., speech). In some embodiments, the diaphragm translates said voltage signal into sound via the neck of a user or via an oral tube. In some embodiments, the device comprises a capacitive sensor and a evaluation board. In some embodiments, the capacitive sensor comprises a touch sensitive panel (e.g., that a user slides their finger over to control frequency of sound). In some embodiments, the user interface comprises one or more of an on/off switch, a frequency control to control the overall frequency range (e.g., male or female) and a volume control. In some embodiments, the user interface, pulse generator circuit and sound source unit are integrated into a single unit. In other embodiments, they are provided on one or more separate units. In some embodiments, the touch sensitive panel controls frequency and or frequency and volume. In some embodiments, the user interface comprises one or more controls selected from, for example a volume control, an overall frequency range control or an on/off switch.

Additional embodiments are described herein.

### DESCRIPTION OF THE FIGURES

FIG. 1 shows the head and neck before and after laryngectomy.

FIG. 2 shows the head and neck with electrolaynx device.

FIG. 3 shows a capacitive evaluation board at low (a) and high (b) positions.

FIG. 4 shows a diagram of input and output frequencies of an exemplary device of embodiments of the present invention.

FIG. 5 shows a diagram of a Darlington circuit used in embodiments of the present invention.

FIG. 6 shows a comparison of the original voltage signal of the Servox EL (FIG. 6A) and the signal produced by a device of embodiments of the present invention (FIG. 6B).

FIG. 7 shows a comparison of the frequency output of the Servox EL (FIG. 7A) and the signal produced by a device of embodiments of the present invention (FIG. 7B).

FIG. 8 shows a comparison of the frequency output of normal voice and the signal produced by a device of embodiments of the present invention.

FIG. 9 shows a photograph of an exemplary device of embodiments of the present invention.

FIG. 10 shows a block diagram of a Servox device.

FIG. 11 shows a block diagram of an exemplary device of embodiments of the present invention.

FIG. 12 shows exemplary C code utilized in embodiments of the present invention.

FIG. 13 shows a line drawing of an exemplary device of embodiments of the present invention.

FIG. 14 shows a line drawing of an exemplary device of embodiments of the present invention.

FIG. 15 shows a line drawing of an exemplary device of embodiments of the present invention.

FIG. 16 shows a line drawing of an exemplary device of embodiments of the present invention.

### DETAILED DESCRIPTION

The present invention relates to electrolarynx devices and their use. In particular, the present invention relates to methods and compositions (e.g., devices) to provide electrolarynx (EL) users with greater intonation in their speech.

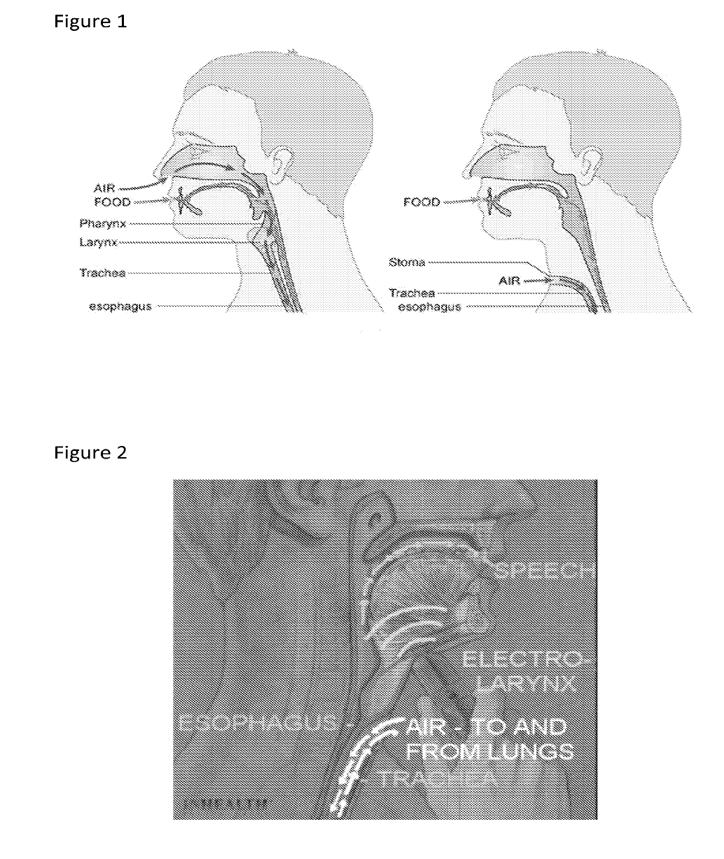

FIGS. 1 and 2 illustrate the head and neck before and after laryngectomy. Intonation, the rise and fall of a voice's pitch, conveys a significant amount of information in speech and has been demonstrated to contribute to speech's intelligibility. Embodiments of the present invention describes devices and methods that deliver greater intonation capabilities to EL users; this design change was realized via 1) the development of an interface that allows the user to control intonation in real-time and 2) electrical circuitry that produces a changing vibration in the EL diaphragm so that the user's desired intonation can manifest as audible speech.

Two current EL models, the Servox® and the TruTone™, were closely evaluated in order to illuminate deficiencies in the designs. The Servox EL uses an interface that consists of two binary buttons to produce either a low or high frequency of speech. These two frequencies are clearly insufficient to model the continuous frequency range of a normal human voice. The Servox EL also has a slide wheel which is used to adjust the volume of the EL speech; however, this wheel cannot be adjusted easily while one of the buttons is being pressed, so the resulting phonation has a constant loudness.

The TruTone design includes a pressure-sensitive button that translates finger pressure into a corresponding frequency along a continuous range. Since the release of the button corresponds to a drop in pressure—and thus a lower frequency—the end of each phonation must drop in pitch; certain phrases—like questions, which often rise in pitch at the end—may be misinterpreted. Like that of the Servox model, the TruTone's volume wheel does not invite real-time adjustment during speech. Thus, neither the Servox nor the TruTone model provides the user with complete control of the speech's intonation.

Accordingly, embodiments of the present invention provide an EL that provides complete control of intonation, as the ability to 1) begin and end phonation at any frequency within an appropriate range of frequencies and 2) change the frequency of the speech in any manner—continuously or discontinuously—and in real-time throughout the entire phonation. These criteria were deemed to model the intonation abilities of normal human speech.

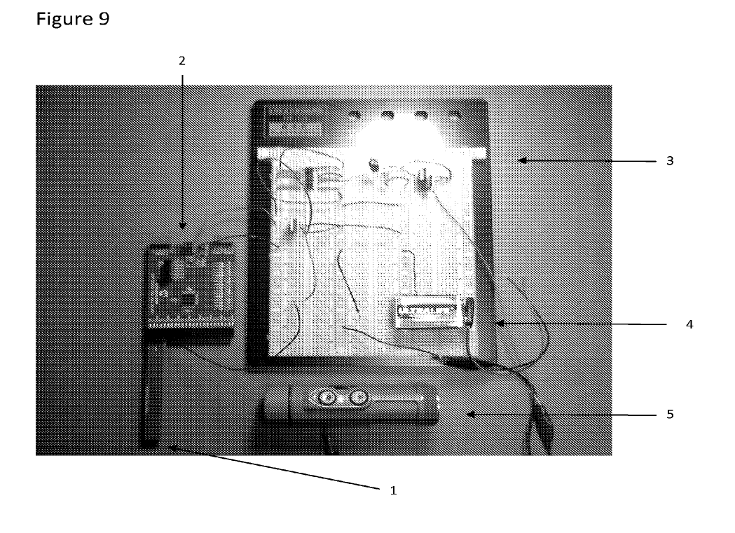

FIG. 9 provides an image of an exemplary device of embodiments of the present invention. The image displays the capacitive sensor 1, evaluation board 2, SN7476 chip 3, Darlington module, 9V battery 4, and sound source unit with its housing 5.

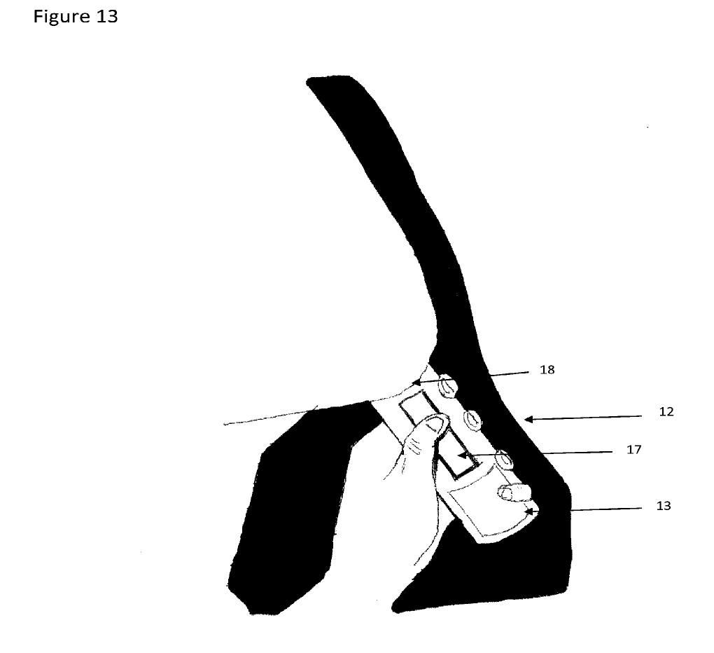

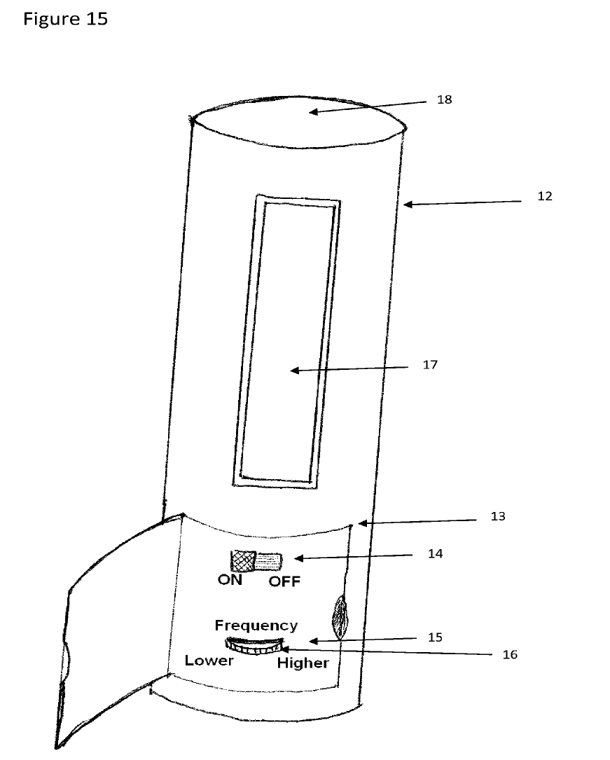

In some embodiments, the user interface and frequency and volume controllers are integrated into the sound source unit as shown in FIGS. 13-15. In other embodiments, they are provided on a separate unit.

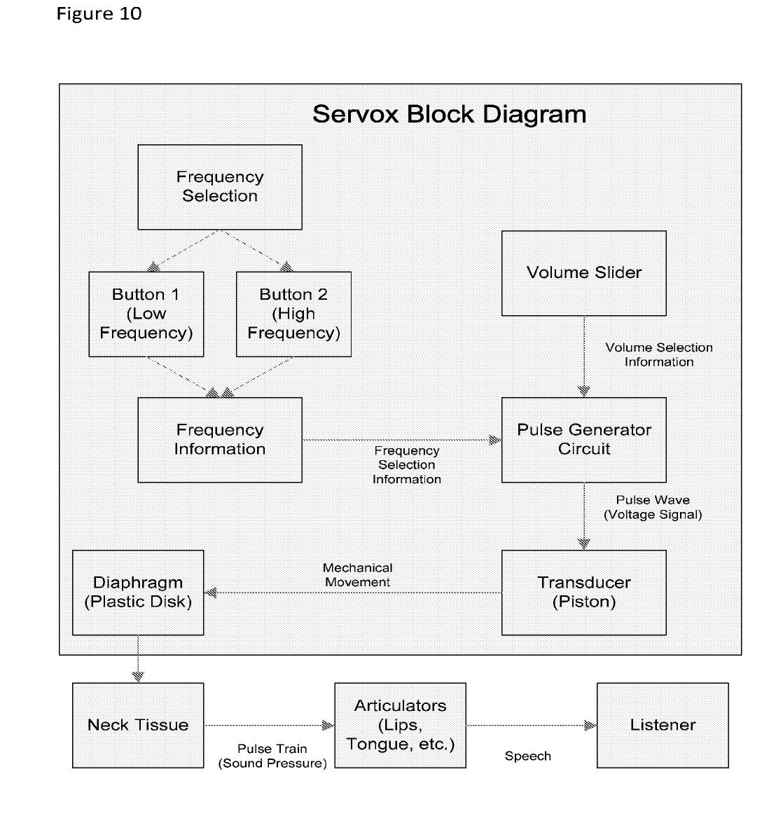

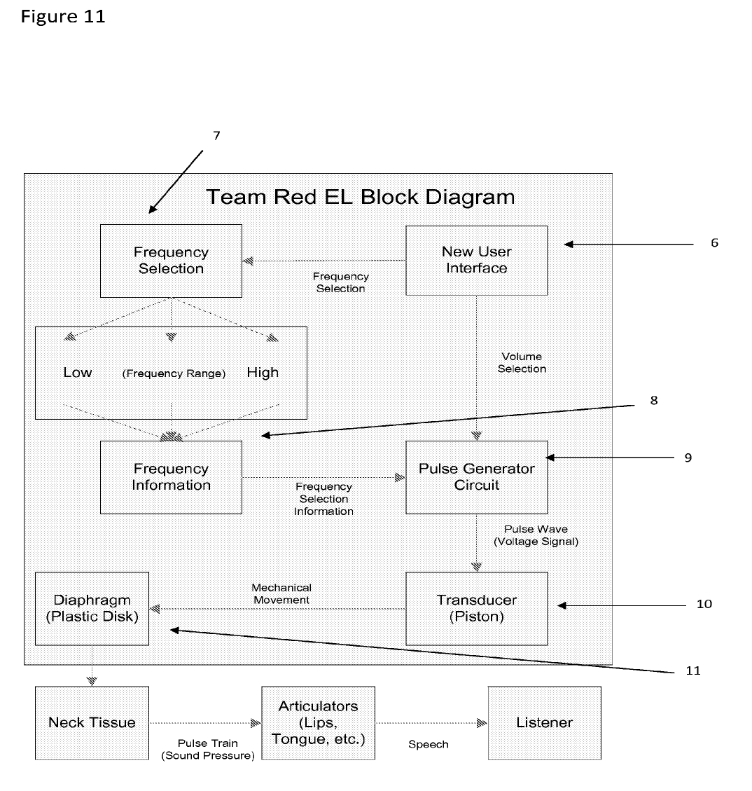

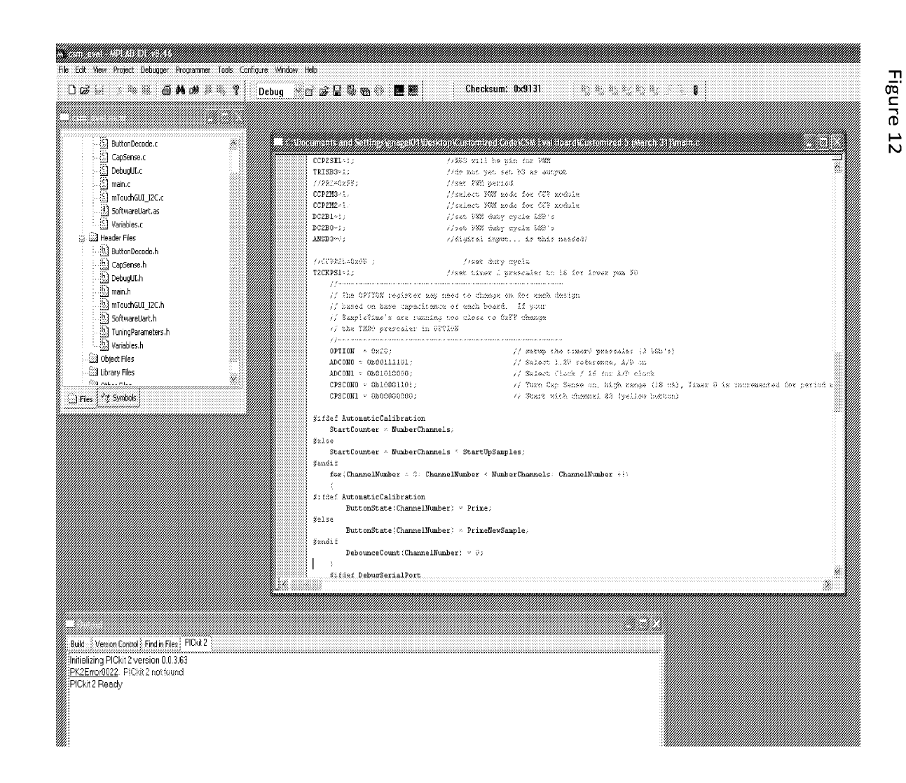

A comparison of the block diagram representations of the Servox and prototype designs also illustrates the improved functionality of the prototype. See FIG. 10 and FIG. 11 for a comparison of the signal flow in the two designs and how they impact the intonation of the EL speech. FIG. 11 shows a block diagram of the device of embodiments of the present invention. The user interface 6 is used to select a frequency 7 and volume. Frequency information 8 is transmitted to a pulse generator circuit 9, a transducer 10 and a diaphragm 11. Sound is then transmitted through neck tissue or a oral tube and then transmitted to the listener.

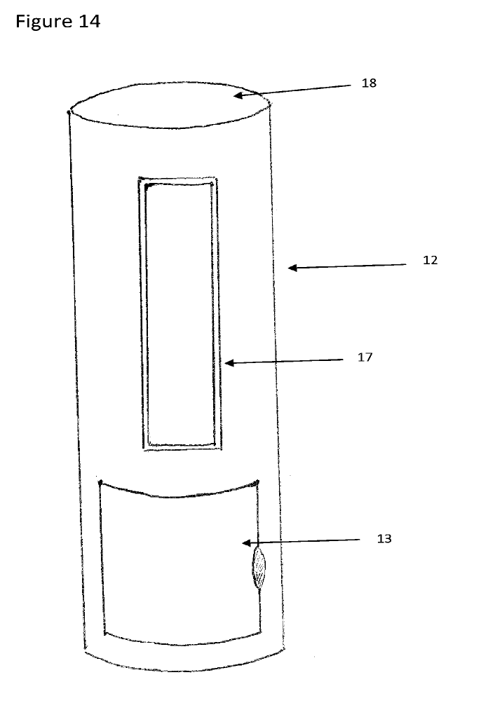

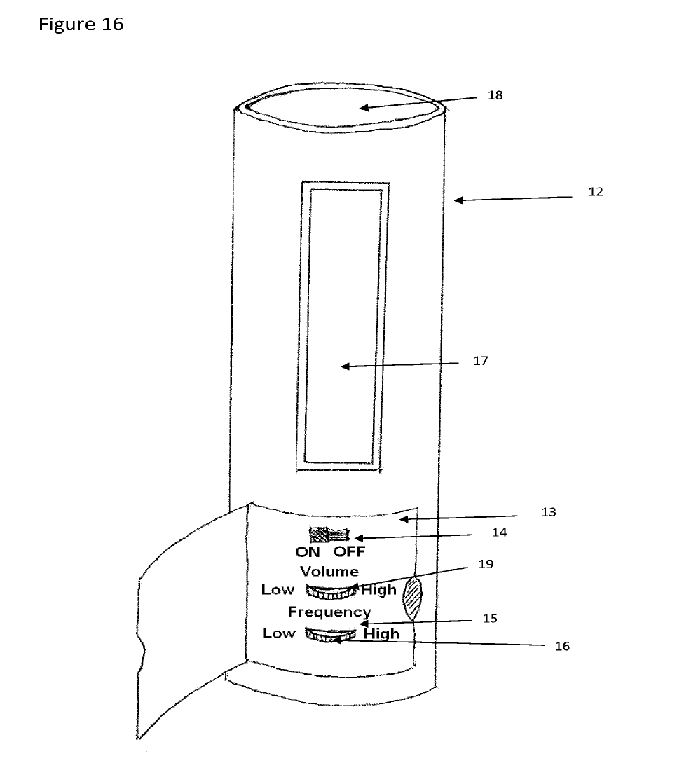

FIG. 13 shows an illustration of an exemplary device 12 held against a user's neck. FIGS. 14-16 show line drawings of exemplary devices 12 of embodiments of the present invention. The drawings show the user interface 13 comprising “on off” switch 14, capacitive sensor 17, diaphragm 18, optional volume control 19 and optional frequency control 15. In some embodiments, the frequency control 15 comprises a frequency selection dial 16 that controls the overall frequency range (e.g., male or female) of the output signal.

To use the device the user moves the “on off” switch 14 to the “on” position, selects a frequency range using the dial 16 and volume using dial 19, places the diaphragm of the device 18 against the neck as shown in FIG. 13 and speaks. The capacitive sensor 17 is used to adjust the frequency of speech as the user is speaking. In some embodiments, the volume dial is set by the user to control the maximum volume of their speech (e.g., depending on whether the user is in a loud or quiet environment). Finger pressure on the capacitive slider is then used as a means of adding inflection to speech and emphasizing certain words or phrases in real-time (as is not possible with current devices). In other embodiments, the capacitive sensor controls only frequency and the volume is controlled with the dial.

Most EL are made to be used by holding them against the outside of the neck, but some have oral adapters, particularly useful when the throat is swollen or sensitive. For example, in some embodiments, a silicone or plastic tube is inserted into a small hole on the mostly closed end of a round rubber silicone or plastic device that looks like a crutch tip. The large open end then is put and pressed over the end of the EL. The user holds the EL up and inserts the tube into the side of the mouth and pushes the EL button to start and stop the sound.

In some embodiments, EL devices are battery powered (e.g., using disposable or rechargeable batteries).

### Experimental

The following examples are provided in order to demonstrate and further illustrate certain preferred embodiments and aspects of the present invention and are not to be construed as limiting the scope thereof.

### EXAMPLE 1

### Electrolarynx Device

### Materials

MICROCHIP mTouch Capacitive Evaluation Kit
SN7476 dual J-K flip-flop
Resistors
Capacitor
TIP31A NPN Transistors
9V Battery
Parts from existing Servox EL
Computer

### Design Process

A modular design strategy was used. The first task was to develop an interface that would allow the user to control the intonation of the EL speech in real-time. A capacitive sensor was used to map finger position to a corresponding frequency of speech.

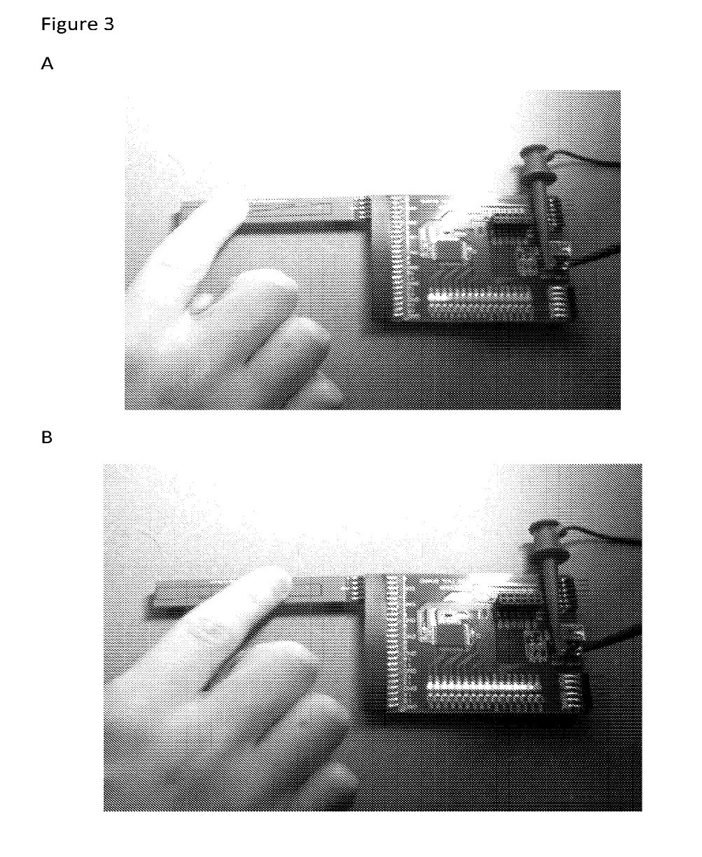

The mTouch Capacitive Evaluation Kit, which contained the capacitive sensor and microcontroller used in the final prototype, includes C code with instructions that allow the sensor to function in a rudimentary fashion. The initial program tracks a finger's position along the capacitive sensor and lights a corresponding number of LEDs on the Capacitive Evaluation Kit's evaluation board to display the finger's position. Examples of the sensor's initial functionality are illustrated in FIG. 3. The low number of lit LEDs corresponds to the finger's position at the bottom of the slider. The high number of lit LEDs corresponds to the finger's position at the top of the slider.

In order to translate the EL user's finger position on the slider to a specific frequency of speech, the C code that was provided with the Capacitive Evaluation Kit was modified. Additional code was added to the program so that the pulse wave generator module of the evaluation board would output a pulse wave of a frequency corresponding to a finger's position on the slider.

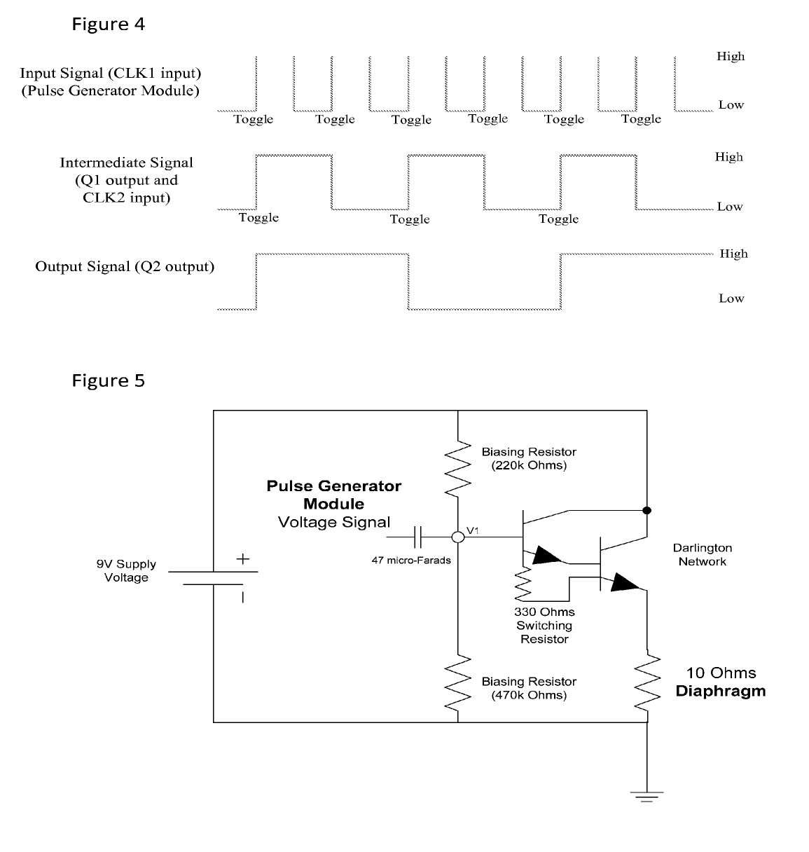

The pulse wave generator of the evaluation board cannot produce a frequency low enough for this particular application. A frequency-quartering circuit was implemented using a SN7476 dual J-K flip-flop chip. The chip was wired so that both flip-flops operate in toggle mode. All of the chip's input and control pins were connected to V_{cc} except for the two clock signals. The output of the evaluation board's pulse wave generator was connected to the clock input of the first flip-flop and the output of the first flip-flop was connected to the clock input of the second flip-flop. The output of the second flip-flop therefore has a frequency equal to exactly one quarter of the input frequency. FIG. 4 shows an illustration of this result.

The diaphragm structure and connected housing of an existing Servox EL was harvested for the construction of a prototype. All existing circuitry was removed from the Servox EL.

The diaphragm of the EL works much like a loudspeaker; current passing through a coil of wire in the presence of a magnet causes the movement of a piston that is related to the magnitude of the current. Because the impedance of the coil of wire in the Servox diaphragm is so small—only 10 ohms—a Darlington transistor arrangement was implemented so that an appropriate DC offset could be introduced in the signal and sufficient current would be provided to the coil. The Darlington circuit that is used in this design is depicted in FIG. 5. The Pulse Generator Module above produces the output signal shown in FIG. 4.

### Results

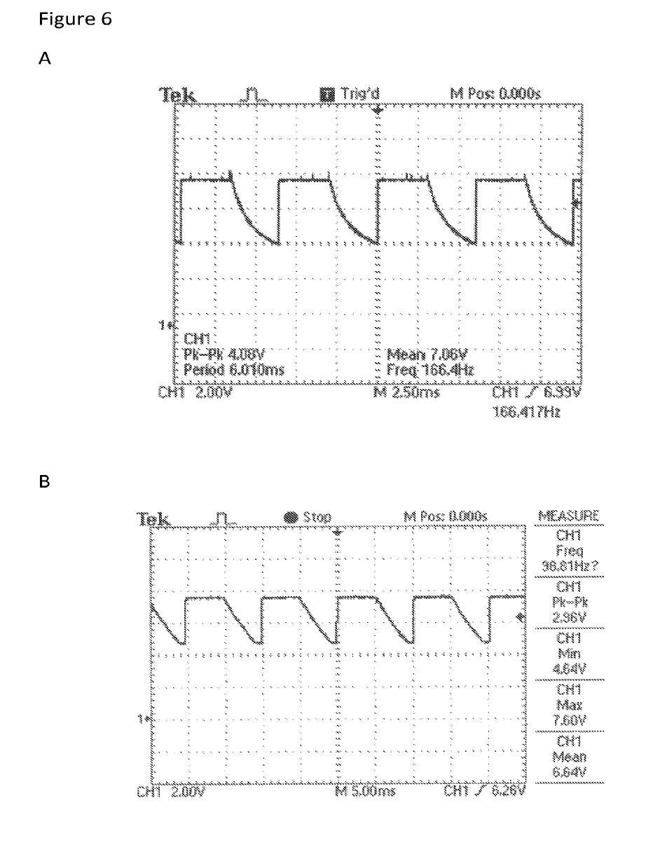

The voltage signal that was produced by the original circuitry of the Servox EL was accurately modeled by the new design. FIG. 6 provides a comparison of the original voltage signal of the Servox EL (FIG. 6A) and the signal produced by the prototype (FIG. 6B).

Matching the DC offset of the waveform produced by the original Servox circuitry ensures that the piston oscillates at an appropriate distance away from the diaphragm; thus, the electro-mechanical transduction operates efficiently and sufficient mechanical energy is delivered to the vocal tract.

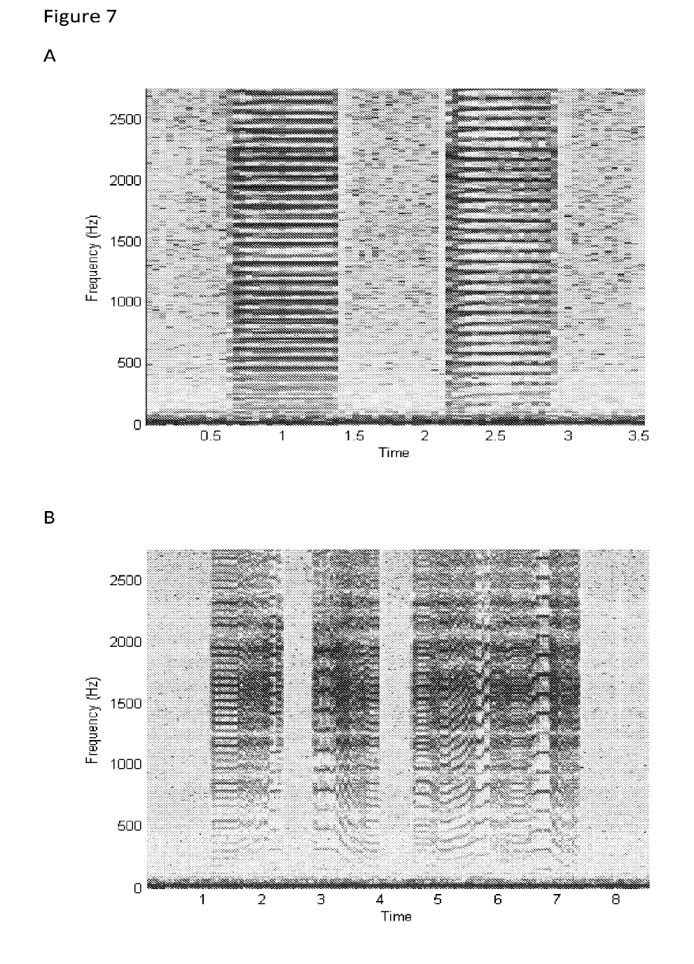

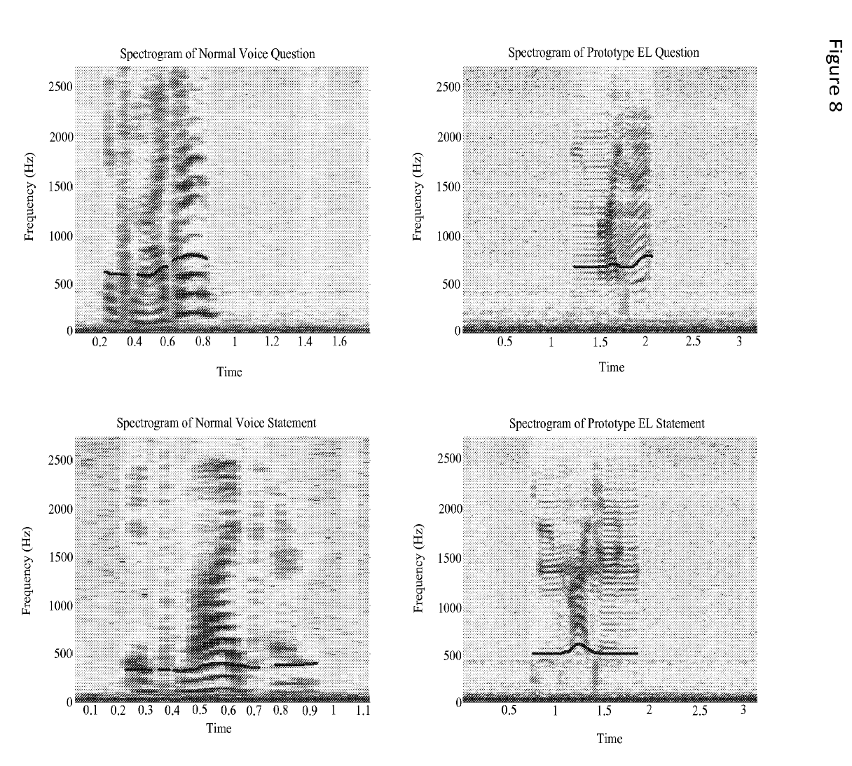

Spectrogram comparisons of the Servox EL and the prototype clearly demonstrate the superior intonation capabilities of the prototype. FIGS. 7-8 illustrate how the Servox (FIG. 7A) can produce only two frequencies, while the prototype (FIG. 7B) is able to change frequencies continuously within a certain range.

### Analysis

The completed prototype produces EL speech with significantly improved intonation compared to the original Servox design. Since the prototype is able to begin and end phonation at any frequency within the desired range, it is also superior to the TruTone model, which must begin and end phonation with a drop in frequency. In some embodiments, components are miniaturized so that all of the circuitry can fit within the EL housing and the capacitive sensor can be mounted on the housing.

In some embodiments, a hardware interface is implemented to allow the user to choose the frequency range over which the EL operates. For example, if the EL is configured to operate in a male's frequency range, a simple adjustment of a slide wheel or comparable dial configures the EL to operate in a female's frequency range. In some embodiments, the capacitive sensor is configured so that it monitors not only a finger's position, but also its pressure on the sensor. With this added functionality, the finger's position could be translated to a frequency as it is now, and the pressure could be translated to a corresponding volume. This would allow the user to speak with even more expression.

### EXAMPLE 2

### Guide to Programming and Implementing the Capacitive Sensor

### Materials

Microchip development tools mtouch capacitive evaluation kit
CapTouch CSM Evaluation Board
CapTouch CTMU Eval Board
4-channel slider plug-in board
2-channel slider
8 button matrix
12 button matrix plug-in board
Pickit Serial Analyzer
mTouch Cap Touch Sense Evaluation Kit CD-ROM
Pickit2 Microcontroller programmer
   (Note that both items can be purchased online through Microchip's website)

### Compiler

For this project, the HI-TECH C Compiler for PIC10-12-16 MCUs V9.70 was used. The most recent version of this software can be found on the HI-TECH website. In order to use the 2-channel slider code, the more efficient PRO mode was used to save space in memory for the 2-channel slider code.

### MPLAB

MPLAB IDE is the MICROCHIP proprietary development environment. This software is used to modify C code that is provided with the capacitive development kit. It can also be used to assemble files to create programmable HEX files. The most recent version can be found on MICROCHIP's website. Once MPLAB IDE is installed, locate the CSM Eval Board folder in the mTouch Cap Touch Sense Evaluation Kit CD-ROM. Extract the CSM_EVAL_Board_Firmware folder onto the desktop. In this folder, there are two csm_eval files. One is the project file, and one is a workspace file. The larger of the two is the workspace file. Double-click on this file to open the workspace with all the files that are pertinent to the project pre-loaded in MPLAB IDE.

In the top-left window of FIG. 12, all of the C-code files pertaining to the project are listed as part of the workspace. The top-center window in the figure displays the C-code file that is currently open and available for modification. Note that multiple files may be opened in the manner simultaneously. The bottom-left window shows the Output display, where the build and program status of the compiler and programmer are shown.

### Compiling and Programming the Microcontroller

To compile the original code given on the CD-Rom, click Project>Build. This command assembles the code in the workspace into a usable hex file.

To program this hex-file onto the CSM Eval board, connect the PICkit2 to the computer via the provided USB cable. Connect the PICkit2 pins of the SCM Eval board to the PICkit2. The PICkit2 programmer should be selected in MPLAB.

Click Programmer>Program to load the hex file onto the microcontroller. The functionality provided by the C-code is now saved on the CSM eval board and can be implemented by providing the board with the necessary supply voltage (4.2V) and connecting the appropriate plug-in board.

2-channel slider

The default C-code provided is configured to provide a one-to-one correspondence between capacitive buttons pressed and the number of LEDs lit on the Eval Board. In the code, this is referred to as BUTTON_ONE_TO_ONE. In order to change the code to function for a 2-channel slider as it pertains to the project, open ButtonDecode.h from the list of files in MPLAB. Comment out line 34, which inactivates the BUTTON_ONE_TO_ONE mode. Un-comment out line 36, which configures the program to be compatible with the 2-channel slider. Now if the code is compiled and programed, the number of LEDs lit should correspond to the position of the finger along the slider. The 2-channel slider must be connected to the Eval board so that the “0” and “1” pins of the slider are connected to the “0” and “1” slots of the eval board.

### Explanation of Code Changes for Capacitive Evaluation Kit Evaluation Board

As explained above, the code that was provided with the Capacitive Evluation Kit was modified so that the finger's position on the sensor could be mapped to an output frequency that would then drive the diaphragm of the EL.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. An electric artificial larynx device, comprising
a) a user interface for selecting a volume and a frequency, wherein said frequency is selected across a frequency range;
b) a pulse generator circuit that translates said volume and frequency into a voltage signal; and
c) a sound source unit comprising a diaphragm that translates said voltage signal into sound.

2. The device of claim 1, wherein said diaphragm translates said voltage signal into sound via the neck of a user.

3. The device of claim 1, wherein said diaphragm translates said voltage signal into sound via an oral tube.

4. The device of claim 1, wherein said user interface comprises a capacitive sensor and a evaluation board.

5. The device of claim 1, wherein said user interface, pulse generator circuit and sound source unit are integrated into a single unit.

6. The device of claim 4, wherein said capacitive sensor comprises a touch sensitive panel.

7. The device of claim 6, wherein said touch sensitive panel controls frequency.

8. The device of claim 7, wherein said touch sensitive panel controls frequency and volume.

9. The device of claim 1, wherein said user interface comprises one or more controls selected from the group consisting of a volume control, an overall frequency range control and an on/off switch.

10. A method, comprising:
a) providing an electric artificial larynx device to a user, said device comprising: i) a user interface for selecting a volume and a frequency, wherein said frequency is selected across a frequency range; ii) a pulse generator circuit that translates said volume and frequency into a voltage signal; and iii) a sound source unit comprising a diaphragm that translates said voltage signal into sound to a subject;
b) selecting a sound and a frequency with said user interface, wherein said frequency is selected across a frequency range; and
c) generating spoken speech with said sound source unit.

11. The method of claim 10, wherein said subject lacks a functional larynx.

12. The method of claim 10, wherein said diaphragm translates said voltage signal into sound via the neck of a user.

13. The method of claim 10, wherein said diaphragm translates said voltage signal into sound via an oral tube.

14. The method of claim 10, wherein said user interface comprises a capacitive sensor and a evaluation board.

15. The method of claim 10, wherein said user interface, pulse generator circuit and sound source unit are integrated into a single unit.

16. The method of claim 14, wherein said user operative said capacitive sensor using a touch sensitive panel.

17. The method of claim 16, wherein said touch sensitive panel controls frequency.

18. The method of claim 17, wherein said touch sensitive panel controls frequency and volume.

19. The method of claim 10, wherein said user interface comprises one or more controls selected from the group consisting of a volume control, an overall frequency range control and an on/off switch.
